(19) United States
(12) Patent Application Publication
(10) Pub. No.: US 2019/0099091 A1
(43) Pub. Date: Apr. 4, 2019

(54) BLOOD PRESSURE MONITOR DEVICE AND METHOD

(71) Applicant: New Vision Telemedicine and Medical Consultant Inc., San Luis Obispo, CA (US)

(72) Inventors: Ahmad Nooristani, San Luis Obispo, CA (US); Lonny Rollins, San Luis Obispo, CA (US)

(21) Appl. No.: 16/135,441

(22) Filed: Sep. 19, 2018

Related U.S. Application Data
(60) Provisional application No. 62/567,119, filed on Oct. 2, 2017.

(51) Int. Cl. A61B 5/022; A61B 5/00; G16H 80/00; A61B 5/0235

(52) U.S. Cl. CPC A61B 5/02233; A61B 5/0004; A61B 5/02141; A61B 5/0022; A61B 5/0235; G16H 80/00

(57) ABSTRACT

The present invention is system for measuring, monitoring and management of a user's physiological condition(s). Measurement data for the user's physiological conditions are stored in an electronic memory and can be transmitted to a central processing network, a physician and emergency medical services. The central processing network contains one or more central processors with algorithms that analyze the user's physiological measurement data by comparison to established thresholds in order to determine the systolic and diastolic pressures of the user.

### CROSS REFERENCE TO RELATED APPLICATION

This application claims priority to U.S. Provisional Application No. 62/567,119, “Vitals Monitor Device and Method” filed Oct. 2, 2017 which is incorporated by reference in its entirety.

### BACKGROUND

There is currently no single all-in-one easy-to-use device for the noninvasive simultaneous measurement of one or more physiological conditions that can be used at home, outside the home or in a medical treatment facility by skilled and unskilled operators and made available to consumers either over-the-counter or by prescription. Medical patients being treated for a variety of symptoms are currently required to physically return to the office of a healthcare provider for measurement of one or more physiological conditions using one or more measurement devices. In addition, in some remote areas of the world, medical practitioners and medical treatment facilities are not conveniently located. In these areas, lack of transportation means and distance to a medical treatment facility are prohibitive so that patients often go without the measurement of physiological conditions and a resulting treatment plan. The necessity for a patient to physically visit a medical treatment facility for routine measurement of physiological conditions have detrimental effects including; loss of time for both the patient and practitioner, increased healthcare cost, reduced opportunity for data collection at strategic intervals, reduced opportunity for patient and practitioner interaction, lack of patient collaboration in the treatment plan and proactive control of their own health and lack of interconnectivity with other devices, personnel, systems and processes.

Patients being treated for a variety of medical conditions are required to return to the physical location of a healthcare provider for the measurement of one or more physiologic conditions as part of their healthcare treatment regime. This can be prohibitive, costly and time consuming for patients with limited financial, mobility and transportation means or who reside in remote rural locations. In some regions of the world healthcare infrastructure is underdeveloped and there are economic, geographic and political barriers to care. If a patient's vital signs are not monitored as prescribed by a healthcare provider with adherence to a corresponding health treatment plan there may be deleterious effects to patient health and outcome. The necessity for patient visitation to a healthcare facility for measurement of vital signs may not be possible, is burdensome, causes inefficiencies including loss of time for both the patient and the healthcare provider, leads to increased cancellation rates, reduces opportunity for data collection at strategic intervals and reduces patient interaction, education and proactive collaboration in a healthcare treatment plan.

Many commercially available remote vital sign monitors are too expensive for broad acceptance in the marketplace. Other devices do not have connectivity and integration into a centralized monitoring and support network. The present invention allows vital signs to be remotely measured and monitored and provides immediate feedback regarding the measurement data without requiring visitation to a medical practice.

What is needed is a single system comprised of one or more measurement modules that can be used at home, outside the home or in a medical treatment facility by skilled and unskilled operators for the safe and effective noninvasive simultaneous measurement of one or more physiological conditions.

### SUMMARY OF THE INVENTION

The present invention is an electromechanical transceiver comprised of one or more electronic and electromechanical components including, but not limited to: a housing containing power, processing and control means, one or more connected sensor assemblies, a display, a user input means, a data transmitting and receiving means, an external device attachment means, software and firmware for the measurement, processing, storage, transmission and receipt of data regarding one or more physiological conditions and one or more of an audio and imaging transmitting and receiving means. A user can be a patient under active care for one or more physiological conditions or a person who monitors one or more physiological conditions as part of their healthcare regimen. The one or more physiological conditions to be measured include, but are not limited to: blood pressure, body temperature, pulse rate, respiration rate, pulse oximetry, blood glucose, electrocardiogram, weight and motion. Measurement data, prompts, alerts and inquiries regarding these conditions can be viewed by a user.

The present invention solves these problems by providing a single system comprised of one or more measurement modules that can be used at home, outside the home or in a medical treatment facility by skilled and unskilled operators for the safe and effective noninvasive simultaneous measurement of one or more physiological conditions. The present invention proves a fast and simple way to test the user's blood pressure and it is anticipated that proper use of the device and close monitoring of the user's condition will greatly reduce the rate of readmission for patients discharged from the hospital. The present invention can be used at a remote field location, where medical practitioners and treatment facilities are not available, by measuring, storing and transmitting data to a medical practitioner by various means including, but not limited to, email, cell, satellite, Bluetooth, and removable storage devices.

The inventive system can be coupled to an adjustable blood pressure sleeve that has an internal bladder which can be coupled to an air pump, a pressure sensor and an orifice. The sleeve can be placed on a limb of a user and adjusted to fit around the circumference of the limb. The air pump can be started which can inflate the internal bladder of the sleeve with air. When the air pressure has reached a sufficiently high predetermined pressure value such as between 180 to 240 millimeters of mercury (mmHg) as detected by the pressure sensor, the processor stops the air pump. When the bladder pressure exceeds the systolic pressure of the user, blood flow through an artery in the limb is stopped. Since the high predetermined pressure for the bladder is greater than the systolic pressure, blood flow through an artery in the limb is stopped when the bladder pressure reaches the high predetermined pressure.

Throughout the bladder inflation and deflation process, air can continuously flow out of the bladder through the orifice and the pressure sensor can detect bladder pressure as it decreases. During the inflation and deflation processes air can flow out of the orifice at an air flow speed of Mach 0.5 to 0.7 below the speed of sound Mach 1.0. The pressure sensor data can be recorded in an electronic memory coupled to the processor. Once the diastolic pressure is reached as detected which can be as low as 40 mmHg, by the pressure sensor no additional information is needed. Once the diastolic value has been determined or the bladder pressure drops below a low predetermined pressure, the pressure relief valve can be opened to quickly and completely depressurize the bladder to ambient pressure.

The bladder pressure data can be recorded and the processor can then analyze the recorded pressure data. From the high predetermined pressure, the detected pressure can initially smoothly decrease over time. When the systolic pressure is reached, blood will start to flow through the artery and the detected bladder pressure can temporarily increase during heart beats and subsequently decrease between heart beats as the bladder pressure continues to decrease. The pressure sensor will eventually detect the pressure at which the temporary increases in detected bladder pressure cease to be detectable which is the diastolic pressure when blood flows continuously through the artery in an expanded state.

In an embodiment, the processor can review the recorded blood pressure data readings from the pressure sensor. The blood pressure data can be recorded as raw data or graphically represented by a graph of pressure y-axis v. time x-axis. Because the pressure sensor can detect very small pressure variations, the pressure sensor readings can be analyzed to very accurately determine magnitudes of each temporary pressure increase as well as the systolic and diastolic pressures. The system can identify and record each of the temporary pressure increases between the systolic and diastolic pressures. The magnitude of a temporary pressure increase can be identified as the differential pressure from the pressure reading prior to a pressure increase to the subsequent pressure increase apex. The time periods between the detected heart beats can be used to calculate the user's heart rate.

While it is possible to use the unfiltered raw data to determine the systolic and diastolic pressures, this can result in errors due to erroneous pressure increases caused by movement or bumping of the bladder during the pressure readings. In an embodiment, the processor can record each temporary pressure increase and identify the magnitudes of each of the temporary pressure increases. The processor can be configured to quantify the first and second highest magnitude pressure increases. The system can use the magnitude of the second highest temporary pressure increase as a data point for filtering the other detected temporary pressure increases. Removal of the highest magnitude pressure increase can remove outlier pressure increases due to bumps or other irregularities. In an embodiment, the processor can take the log (base 10) of the second highest magnitude pressure increase. For example, in an embodiment, the systolic threshold can be set to 27% of the first or second highest magnitude pressure increase and the diastolic threshold can be set to 31% of the first or second highest magnitude pressure increase. In an embodiment, any temporary pressure increase below these values will be dismissed and not identified as temporary pressure increase which can be used to identify the pulse rate or the systolic and diastolic pressures. The filtered pressure data can then be used to identify the systolic pressure when a temporary pressure increase is detected and diastolic pressures when the temporary pressure increases are no longer detected. The system processor can identify and output the diastolic and systolic pressures which can be through a visual display, an audio output or other output mechanism.

In an embodiment, the users' physiological measurement data can be securely transmitted, under HIPAA compliant standards, to a central processing network, to the user's healthcare provider and emergency medical services. The present invention allows an individual to take a proactive role in their own healthcare, to increase the frequency of data collection and analysis by a central processing network and user physician. It allows a user's physician to actively and remotely monitor a user's vital signs and make adjustments to their healthcare plan, without requiring physical visitation, resulting in improved user health and outcome, increased efficiency and reduced cost.

### BRIEF DESCRIPTION OF THE DRAWINGS

FIG. 1 illustrates an embodiment of the user electromechanical transceiver including blood pressure and pulse oximetry assemblies.

FIG. 2 illustrates a block diagram of an embodiment of the system architecture of the user electromechanical transceiver.

FIG. 3 illustrates a block diagram of an embodiment of blood pressure measuring system components.

FIG. 4 illustrates an embodiment of an air flow orifice.

FIG. 5 illustrates an example pressure graph of a blood pressure test of a system user.

FIG. 6 illustrates a portion of an example pressure graph that includes the systolic and diastolic pressures of a system user.

FIG. 7 illustrates a portion of an example pressure graph illustrating a magnitude of a temporary pressure increase.

FIGS. 8a and 8b illustrate flow charts of the processing used to determine the systolic and diastolic pressures of a system user.

FIG. 9 illustrates a diagram of the user electromechanical transceiver connectivity to a central processing network, the user's physician and emergency medical service.

FIG. 10 illustrates examples of prompts, 180/90 alerts and inquires.

FIG. 11 illustrates a diagram of the user electromechanical transceiver connectivity to the central processor in the central processing network.

### DETAILED DESCRIPTION

The terminology used herein describes various embodiments of the present invention and is not intended to be limiting. In describing the invention, it is understood that many techniques and steps are disclosed. Each of these has individual benefit and each can also be used in conjunction with one or more of the other disclosed elements. This disclosure will refrain from repeating every possible combination of individual steps and should be reviewed with the understanding that such combinations are within the scope of the invention and claims. The present disclosure is to be considered as an exemplification of the invention and is not intended to limit the invention to the specific embodiments illustrated by the figures or following descriptions but to satisfy the applicable legal requirements. The present invention is described with reference to the appended figures representing the preferred embodiments.

The present invention is a user wearable electromechanical device for the simultaneous measurement storage, transmission and receipt of data regarding one or more physiological conditions and contains one or more electronic and electromechanical components. The one or more electronic and electromechanical components may include a power source such as a battery or AC to DC power supply, voltage conditioning electronics such as a voltage regulation circuit with a feedback loop, a control and processing electronics system such as a multifunctional microcontroller with a variety of inputs and outputs, sensor assemblies such as a blood pressure cuff, a pulse oximeter, or electrocardiogram sensors, a display such as a color or monochrome touch screen, a user input means such as a touch screen, voice to data, keypads and pushbuttons, an audio recording, storage, transmitting and receiving means including one or more microphones and speakers, an imaging recording, storage, transmitting and receiving means for one or more still and video images, a data transmitting and receiving means including one or more, but not limited to, embedded WiFi, Bluetooth, 3G, 4G and LTE modules and a means for attaching external devices. The one or more physiologic conditions to be measured include one or more, but not limited to, temperature, pulse rate, blood pressure, respiration rate, pulse oximetry, blood glucose, heart rhythm/electrocardiogram, weight and motion. A preferred embodiment of the present invention includes an electromechanical transceiver comprised of a housing preferably manufactured from an injection molded or machined polymer or metal substrate. The housing being constructed to contain one or more electronic and electromechanical components including, but not limited to, a power source, a voltage conditioning, processing and control means, one or more sensor assemblies, a display, a user input means, an audio recording, storage, transmitting and receiving means, an image recording, storage, transmitting and receiving means, a data recording, transmitting and receiving means, a means for attaching one or more external devices and software for the measurement, processing, recording, transmitting and receiving of data, audio, video and images. The display, user input means, audio recording, storage, transmitting and receiving means, and image recording, storage, transmitting and receiving means further comprised of selectable language formats including one or more, but not limited to, English, Spanish, French, German, Chinese, Japanese and Russian.

In an embodiment, the system can initiate blood pressure measurements automatically when the device is correctly worn and will not begin measuring if the device is not correctly worn. The measurement data for one or more physiological conditions may be simultaneously displayed, stored and transmitted from the user electromechanical transceiver and to one or more of a central processing network, the user's physician and emergency medical services.

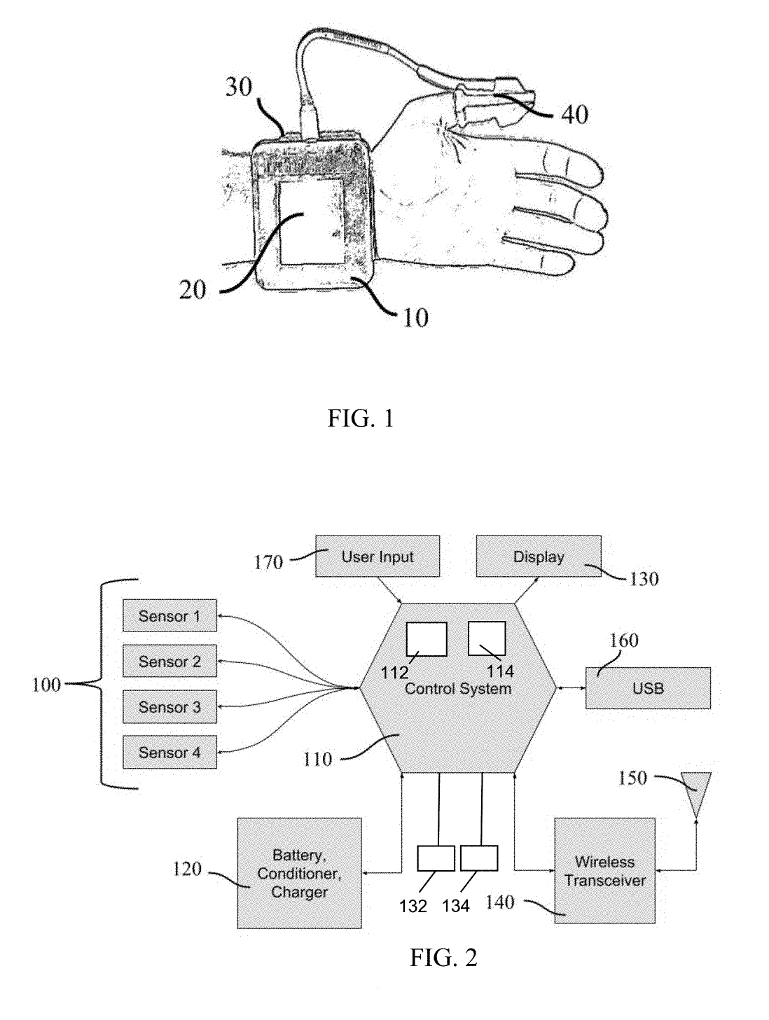

FIG. 1 depicts one embodiment of the present invention where a user physiological measurement transceiver 10 (“transceiver device”) is comprised of a housing containing one or more of a display 20, a blood pressure cuff assembly 30, a fingertip pulse oximeter assembly 40 and power and control electronics including audio and image recording, storage, transmitting and receiving means. In the illustrated embodiment, the blood pressure cuff assembly 30 can be secured around the wrist or forearm of the user. The blood pressure cuff assembly 30 can be an inflatable structure that can be wrapped around the limb of the user. The blood pressure cuff assembly 30 can include hook and loop portions which can allow the cuff assembly 30 be secured around the limb of the user prior to inflation of the cuff assembly 30. In the illustrated embodiment, the fingertip pulse oximeter assembly 40 can be placed on a thumb of the user. In other embodiments, the fingertip pulse oximeter assembly 40 can be placed on any other digit of the user.

The audio means containing one or more, but not limited to, microphones and speakers. The imaging means containing one or more, but not limited to, CCD and CMOS sensors for still and video images. The display, user input means, audio recording, storage, transmitting and receiving means, and image recording, storage, transmitting and receiving means are further comprised of selectable language formats including one or more, but not limited to, English, Spanish, French, German, Chinese, Japanese and Russian.

With reference to FIG. 2 a block diagram of one embodiment of the system components of present invention is illustrated. The system may include one or more sensor assemblies 100, such as a blood pressure cuff assembly, a pulse oximeter assembly, temperature sensor, heart rate sensor, blood oxygen level, etc. The sensors 100 can be electrically connected to the inputs of the control system 110 which can include an electronic memory 114 which can store data from the sensor(s) 100. The processor 112 can process the data from the sensor(s) 100 to provide user physiological measurements. The processor 110 controls an air pump 132, which inflates the bladder up to an upper predetermined pressure and the pressure relief valve 134 which can relieve the pressure from the bladder after a lower predetermined pressure is reached.

The device receives electrical power from a battery, voltage conditioner and charger electronics 120. The control system 110 outputs are used to power the system display 130 and wireless transceiver 140 with global positioning system (GPS) and antenna 150. A USB connector 160 allows external devices such as a power supply to be attached. User input 170, such as a response to inquiries, may be accomplished using one or more methods including, but not limited to, touch screens, keypads and a variety of push buttons and switches.

### General Operation

Blood pressure cuffs and stethoscopes are used to measure blood pressure and listen to heart beats. With reference to FIG. 1, the blood pressure cuff assembly 30 is placed around the limb of the user. In the illustrated embodiment, the blood pressure cuff assembly 30 is placed around the wrist. However, in other embodiments, the blood pressure cuff assembly 30 is placed around the bicep or other portion of a limb of the user. The blood pressure cuff assembly 30 can have a tension control mechanism such as hook and loop mechanisms which allow the blood pressure cuff assembly 30 to be closely secured around the perimeter of the limb prior to inflating the bladder of the blood pressure cuff assembly 30.

With reference to FIG. 3 illustrates a block diagram of the inventive system components used for determining the blood pressure of the user. The blood pressure cuff assembly includes a bladder 170 coupled to an air pump 172, an orifice 174, a pressure sensor 176, and a pressure relief valve 178. After the blood pressure cuff assembly is placed on the limb of the user and secured in place, the air pump 172 is actuated and air is pumped into the bladder 170. The pressure sensor 176 detects the air pressure within the bladder 170.

During this air pumping process, air flows out of the bladder 170 through the orifice 174. The pressure in the bladder 170 can be increased up to a high predetermined pressure which can be detected by the pressure sensor 176 and the pressure information is provided to the processor 175 and can be stored in memory 173. The high predetermined pressure setting needs to be above the systolic pressure of the user so that blood flow through an artery in the limb is stopped but not too much higher because no useful information is obtained at pressures above the systolic pressure. In different embodiments, the high predetermined pressure can be between 180 to 240 mmHg. While systolic blood pressures above 180 to 240 mmHg have been measured, these are extreme cases where immediate medical attention is required. In general, the system may output a message asking the user to seek immediate medical attention if the systolic blood pressure is over 180 mmHg. If the user's systolic blood pressure is above the high predetermined pressure value, the system can output an error message or a systolic blood pressure that matches the high predetermined pressure value.

Once the pressure in the bladder 170 reaches or exceeds the predetermined pressure, the processor 175 causes the air pump 172 to stop and cease inflating the bladder 170. The air pressure will begin to decrease as air continues to flow from the bladder 170 through the orifice 174. The pressure sensor 176 continues to detect the decreasing pressure in the bladder 170 and this pressure data includes information that is used to determine the systolic and diastolic blood pressures of the user. The pressure data from the pressure sensor 176 is stored in the memory 173 and processed by the processor 175. Once the lower predetermined pressure is reached, the processor 175 can cause the pressure relief valve 178 to open to completely vent the bladder 170 pressure. Once the bladder 170 is vented, the data from the pressure sensor 176 will no longer be recorded or stored. The processor 175 can now analyze the stored data to determine the systolic and diastolic pressures.

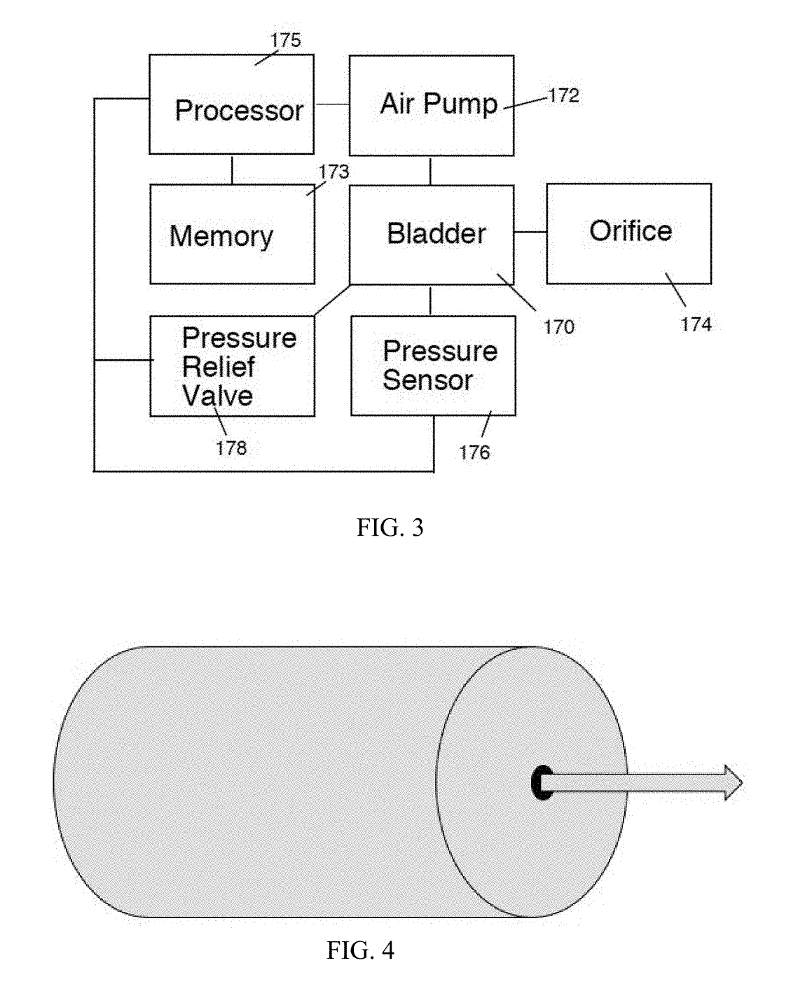

As discussed, air will continue to flow through the orifice 174 which is coupled to the bladder 170 throughout the inflation and deflation processes. An embodiment of the orifice 174 is illustrated in FIG. 4. The orifice 174 can have a diameter that is about 0.008 inches and a depth that is about 0.10 inches. In other embodiments, the orifice 174 can have other dimensions. The air flow through the orifice 174 can be very high but lower than the speed of sound, Mach 1.0. For example, the fastest air flow rate through the orifice 174 can be Mach 0.5 to 0.7. The orifice 174 can function to continuously reduce the bladder 170 pressure. The air flow rate through the orifice 174 can be much lower than the air flow rate of the air pump 172. Thus, when the air pump 172 is on, the air flow rate into the bladder 170 is higher than the air flow rate through the orifice 174 so the air pressure in the bladder 170 increases. The air flow rate through the orifice 174 can control the time duration of the bladder 170 inflation and deflation processes. In an embodiment, the system can obtain the blood pressure readings in a few seconds. For example, the system can obtain the blood pressure readings in as little as 2 seconds. However, the accuracy of the pressure readings can be improved by reducing the bladder 170 pressure at a slower rate which can more preferably be 3.5 seconds or more.

The accuracy of the systolic pressure measurement is based upon the detected heartbeats by the pressure sensor 176 and the detection of the diastolic pressure measurement is based upon the inability to detect heart beats by the pressure sensor 176. When the rate of pressure change is too great, it can be difficult to accurately match the heart beat detection to specific pressures because the rate of pressure change is too high. Similarly, if a system user has a very low heart rate, this can also prevent the accurate detection of the systolic and diastolic pressures. For example, if a user has a 60 beat per minute (BPM) heart rate, there is only 1 heartbeat per second. If the systolic and diastolic pressures are recorded within 5 seconds, there may only be 5 heartbeats available to the inventive system to determine the systolic and diastolic pressures. Because the change in pressure can be about 5-8 mmHg per beat, the accuracy of this pressure analysis can be + or −5-8 mmHg. By slowing the rate of pressure decrease from the bladder 170, the accuracy of the test can be improved because the rate of change in bladder 170 pressure can be much lower between each heartbeat. In different embodiments, the inventive system can be configured to reduce the rate of change in pressure and extend the duration of the pressure decrease to improve the accuracy of the systolic and diastolic pressure measurements.

### Blood Pressuring Monitoring Process

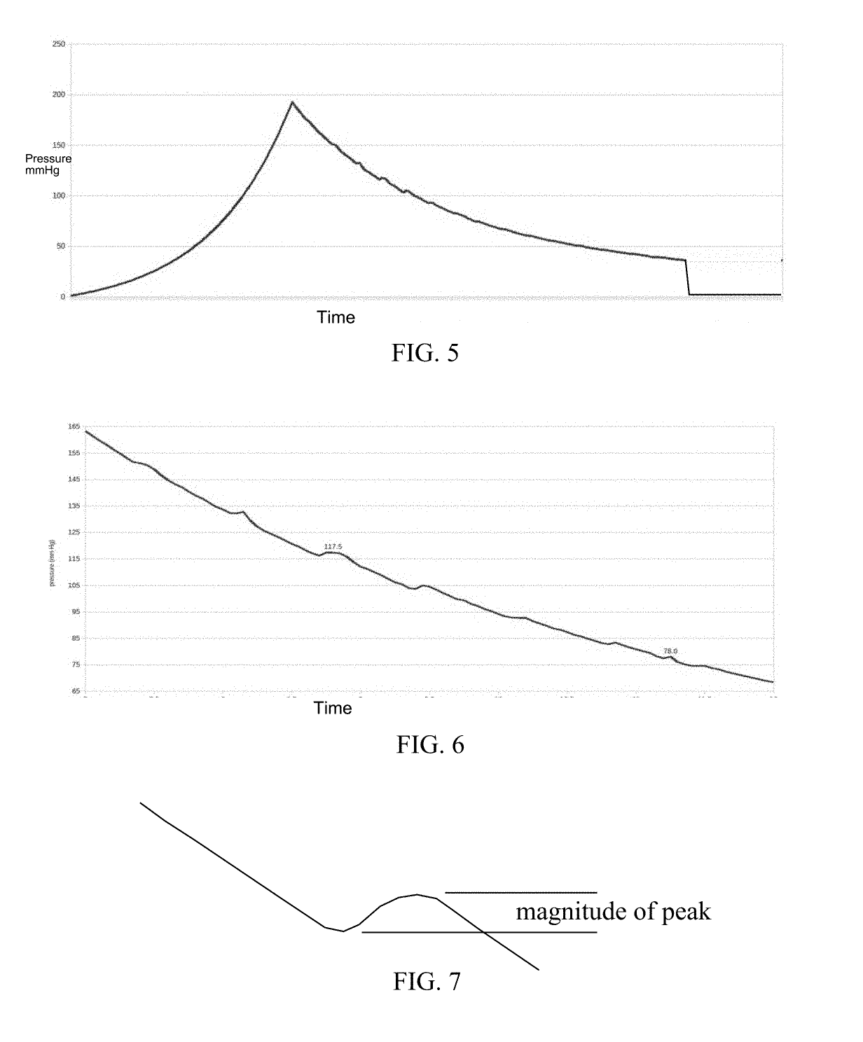

As discussed, once the air pump causes the bladder pressure to reach a high predetermined value, the pump is turned off and air is released at a fixed rate out the orifice. With reference to FIG. 5, a bladder pressure graph is illustrated with the vertical Y-axis as pressure and the X-axis time. In this example, the pressure ramps up from 0 mmHg to about 180 mmHg which can be the high predetermined value when the air pump is turned on and simultaneously air is flowing out of the orifice. The peak of the pressure graph indicates the point at which the air pump is turned off. The pressure then decreases as air flows out of the bladder through the orifice. The pressure line is initially smooth since no temporary pressure increases are detected. When the user's blood starts to flow through an artery at the systolic pressure, the bladder pressure begins to have temporary pressure increases which are shown as small variations in the pressure line. When blood continuously flows through the artery at the diastolic pressure, the temporary pressure increases are no longer detected and the pressure line in the graph become smooth again. Once the pressure reaches the diastolic pressure or the low predetermined pressure, the relief valve can open and the bladder pressure can be immediately taken down to ambient pressure. In the illustrated embodiment, the bladder pressure has steadily dropped to a low predetermined pressure of 40 mmHg at which point the relief valve opens and quickly vents the bladder pressure. Normally, the detected diastolic pressure will be greater than 40 mmHg and the system will open the relief valve at a higher bladder pressure.

With reference to FIG. 6 an enlarged view of an example bladder pressure graph is illustrated which shows the temporary blood pressure peaks. In this example, several temporary blood pressure increases are shown as variations in the pressure line of the graph. In this example, the processor can identify temporary pressure increases any time that an increased subsequent pressure is detected. The system can denote the pressure from the pressure sensor where the first pulse is detected. Since pressure will decrease at a stable rate, any temporary increase can be attributed to a pulse. Once the first pulse is detected, the system can denote this as the systolic value. The system can also identify the pressure at which temporary increases in pressure are no longer detected and denote this as the diastolic pressure.

However, this method may not be accurate. The problem is that the pressure sensor can be very sensitive. For example, a minimum temporary pressure increase change threshold is utilized to eliminate any small limb movements made by the patient which may be improperly interpreted as a pulse. In an embodiment, the system can perform a filtering process on the pressure sensor data to improve the accuracy. More specifically, the filtering can be performed by removing temporary pressure increases that are below a threshold value. In order to perform this filtration, the system can identify the magnitudes of each of the temporary pressure increases. The way the amplitude is determined in a decreasing slope must be reliable. With that said, there are several ways that are acceptable.

In an embodiment, the amplitude of a peak can be defined as the difference between the pressure at the previous valley and the pressure at the peak. A valley is defined as the point at which pressure starts to increase after it has been decreasing. Alternatively, a peak is defined as the point at which pressure starts to decrease after it has been increasing. With reference to FIG. 7, the magnitude of each temporary pressure increase measurement can be obtained by subtracting the pressure just prior to the detected pressure increase from the pressure at the apex of the temporary pressure increase. The system can then review the magnitudes of all of the temporary pressure increases and identify a second largest magnitude temporary pressure increase. In general, the magnitudes of the temporary pressure increases can be similar within a reasonable range. However, if the bladder is bumped during the pressure sensing process, the first largest magnitude can be substantially larger than normal and outside the normal range.

In another embodiment, the system can measure the magnitudes of the temporary pressure increases from below the peak. The amplitude of a peak during a declining slope is determined by drawing a line between the valley just before and just after the peak. The system can then derive the magnitude from the peak to a point on the line directly below the peak. In other embodiments, other methods can be used to determine the magnitudes of the peaks. As long as a consistent method is used, the system will be able to accurately compare the magnitudes of the peaks.

In an embodiment, in order to reduce the possibility of false pulse detections, the system can remove the largest magnitude temporary pressure increase which can substantially improve the likelihood of error. While it is possible that a user may bump the bladder multiple times, this is much less likely within the limited testing time period.

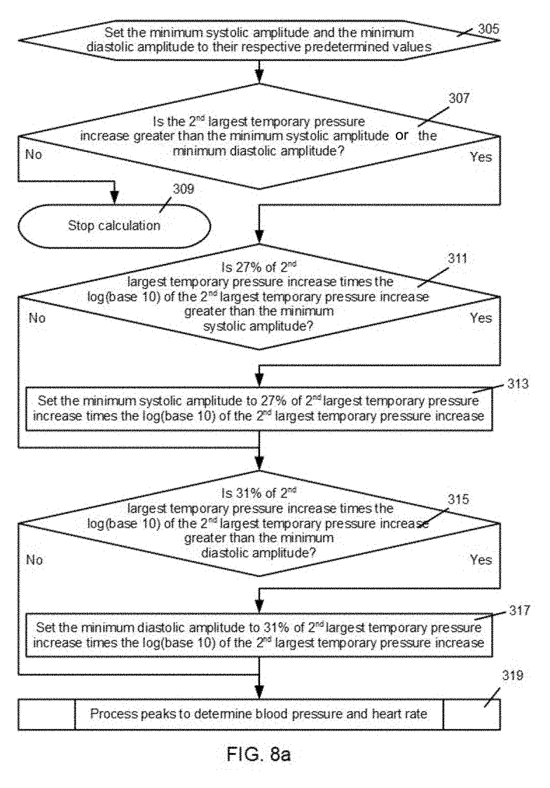

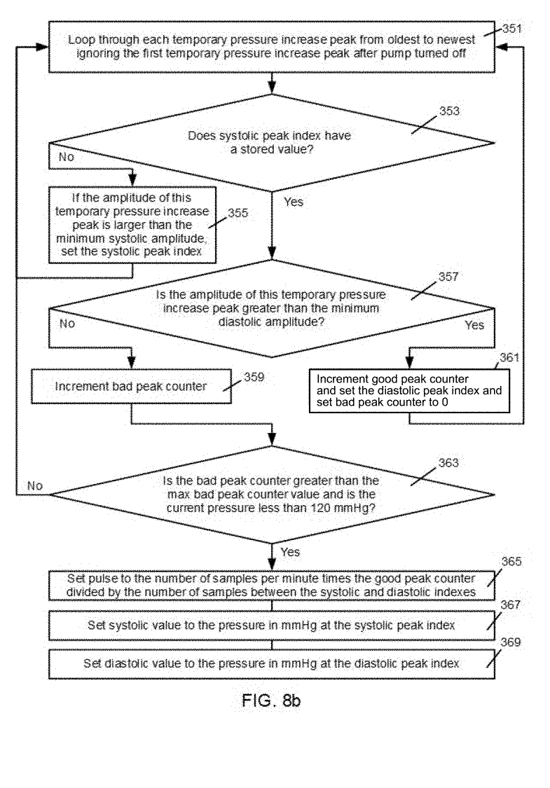

Since the system can detect a much smaller pulse than a standard operator can hear, an entire pressure profile for the test can first be graphed and then a pass/fail criteria is generated by the system. This criteria is then applied to each detected temporary pressure increase to determine which of them are a valid pulse. In an embodiment with reference to FIG. 8a, a flowchart illustrating steps that the system can perform for filtering the temporary pressure increases. The filtering process can begin with a predetermined minimum systolic amplitude and a predetermined minimum diastolic amplitude which can be stored in a memory 305. Once the amplitudes of the temporary pressure increases are received and stored, the system can find the amplitude of 2nd largest pulse and stop the calculation 309 if it is less than the minimum systolic amplitude or the minimum diastolic amplitude 307. If 27% of the 2nd largest temporary pressure increase times the log (base 10) of the 2nd largest measured temporary pressure increase is greater than the predetermined minimum systolic amplitude 311, then this value can be stored as the new minimum systolic amplitude 313. If 31% of the 2nd largest temporary pressure increase times the log (base 10) of the 2nd largest measured temporary pressure increase is greater than the predetermined minimum diastolic amplitude 315, then this value can be stored as the new minimum diastolic amplitude 317. Now that a graph-specific minimum systolic amplitude and a graph-specific minimum diastolic amplitude have been calculated, these values can be used to determine an accurate blood pressure and heart rate 319.

FIG. 8b continues by looping through each detected temporary pressure increase peak from the oldest to the newest but ignores the first temporary pressure increase 351 because the pressure continues to rise after the pump has been turned off and this first temporary pressure increase should not be considered a valid pulse. If the systolic peak index does not already have a stored value 353 and if the amplitude of this peak is greater than the minimum systolic peak amplitude then the system will identify this peak is a valid pulse and the system can set the systolic peak index to the current peak index 355. If the systolic peak index has a stored value 353 and the amplitude of this peak is greater than the minimum diastolic amplitude 357 then this peak is a valid pulse and the system can clear the bad peak counter (set it to zero), increment the good peak counter, and set the diastolic peak index to the current peak index 361. If however, the systolic peak index has a stored value 353 and the amplitude of this peak is not greater than the minimum diastolic amplitude 357 then this peak is not a valid pulse and the system can increment the bad peak counter 359. In this case, if the bad peak counter is greater than a predetermined max bad peak counter and the current pressure is less than 120 mmHg then the system stop looping 363. Once the looping has stopped, then the system can determine the pulse (heart rate) based upon the number of samples taken per minute times the good peak counter divided by the number of samples between the systolic and diastolic indexes 365. The system can set the systolic value to the pressure in mmHg at the systolic peak index 367 and set the diastolic value to the pressure in mmHg at the diastolic peak index 369. The system can then display the pulse in beats per minute along with the systolic and diastolic pressures in mmHg on a visual display.

The data used to create the graph can be an array of pressure readings that is obtained through the described test process. The array counter can be a memory device in communications with a processor that holds the index in the array of the most recent pressure readings. The raw data from the pressure sensor can be obtained by the processor and stored in electronic memory. In an embodiment, a bladder pressure sample measurement can be taken at uniform reading time increments, for example every 50 ms. Anytime the blood pressure process is active, the graph can be evaluated at uniform evaluation time increments, for example every 256 ms. If a diastolic pressure is detected during a graph evaluation, the valve can be immediately opened since the diastolic value is detected and no other bladder pressure information is needed and the patient does not need to wait for the bladder to completely deflate through air flow through the orifice. Every 256 ms., the amplitude of the second largest peak is found in the array. In an embodiment, the system can use the second largest amplitude peak while the first largest amplitude peak can be ignored. The second largest amplitude peak is used so as to filter out any possible erroneous and very large peak. Multiplying this value to its log (base 10) ultimately adds a small amount to smaller values and a larger amount to larger values. For example, if the largest peak is 100, the result would be 200 (+100) and if the largest peak is 1000, the result would be 3000 (+2000).

With reference to Table 1 below, a sequence of bladder pressure sensor values readings from a blood pressure test are listed. These values are directly measured by the pressure sensor every 50 ms. during the bladder increase and decrease process steps. Each of these values can be converted into pressure values with units of mmHg using the equation:

mmHg=(((value*400.125/4096)−20)/9)*7.50062

This equation can be specific to the pressure sensor used by the system. Different pressure sensors can have different outputs and may require different equations to convert the pressure sensor readings into pressure outputs.

TABLE 1
204, 214, 217, 221, 225, 228, 232, 236, 241, 245, 249, 253, 258, 263, 267, 272, 277, 282, 287,
292, 298, 303, 309, 314, 320, 326, 332, 339, 345, 351, 358, 365, 372, 379, 386, 394, 401, 409,
417, 425, 433, 442, 450, 459, 468, 477, 487, 496, 506, 516, 527, 537, 548, 559, 570, 581, 593,
605, 617, 630, 643, 656, 669, 683, 697, 711, 725, 740, 756, 771, 787, 803, 820, 837, 854, 872,
890, 908, 927, 947, 966, 986, 1007, 1028, 1050, 1072, 1094, 1117, 1141, 1165, 1189, 1214,
1240, 1266, 1293, 1320, 1348, 1377, 1406, 1436, 1466, 1498, 1529, 1562, 1595, 1629, 1664,
1700, 1736, 1773, 1811, 1850, 1889, 1930, 1971, 2013, 2057, 2101, 2146, 2192, 2239, 2287,
2337, 2387, 2438, 2491, 2478, 2445, 2415, 2387, 2364, 2335, 2313, 2290, 2272, 2256, 2231,
2209, 2186, 2163, 2144, 2123, 2103, 2084, 2063, 2044, 2023, 2005, 1999, 1991, 1972, 1947,
1924, 1906, 1892, 1873, 1855, 1841, 1821, 1804, 1791, 1775, 1773, 1780, 1741, 1714, 1694,
1683, 1668, 1653, 1637, 1624, 1609, 1594, 1583, 1597, 1598, 1594, 1578, 1554, 1535, 1524,
1510, 1496, 1480, 1464, 1454, 1437, 1433, 1450, 1445, 1430, 1415, 1402, 1388, 1383, 1367,
1357, 1344, 1333, 1321, 1311, 1305, 1304, 1303, 1290, 1279, 1268, 1255, 1250, 1239, 1228,
1220, 1209, 1201, 1190, 1186, 1193, 1181, 1171, 1162, 1154, 1147, 1130, 1121, 1128, 1106,
1095, 1089, 1086, 1089, 1078, 1072, 1061, 1054, 1047, 1041, 1034, 1027, 1020, 1014, 1008,
1002, 1000, 997, 994, 985, 978, 971, 964, 961, 953, 948, 942, 936, 931, 926, 926, 921, 917, 912,
906, 900, 896, 890, 884, 880, 878, 869, 864, 862, 859, 857, 853, 849, 843, 840, 835, 831, 826,
821, 819, 813, 810, 806, 806, 803, 797, 797, 790, 784, 783, 778, 774, 771, 767, 765, 760, 757,
754, 755, 752, 751, 743, 740, 737, 734, 730, 726, 723, 721, 718, 715, 712, 707, 708, 707, 705,
701, 699, 695, 692, 688, 685, 684, 678, 675, 669, 668, 670, 667, 666, 664, 661, 661, 655, 653,
651, 649, 646, 643, 639, 639, 636, 635, 631, 547, 463, 378, 293, 204, 204, 204, 204, 204, 204,
204, 204, 204, 204, 204, 204, 204, 204, 204, 204, 204, 204, 204, 204, 204, 204, 204, 204, 204,
204, 204, 204, 204, 204, 204, 204, 204, 204, 204, 204, 204, 204, 204, 204, 204, 204, 204, 204,
204, 204, 204, 204, 204, 204

Table 2 below lists the pressure readings from Table 1 after they have been converted into pressure in units of mmHg using the conversion equation and rounded to 2 decimal places.

TABLE 2
0.00, 1.29, 1.54, 1.88, 2.22, 2.47, 2.8, 3.14, 3.56, 3.89, 4.23, 4.57, 4.99, 5.41, 5.74, 6.16, 6.58, 7,
7.42, 7.84, 8.34, 8.76, 9.27, 9.69, 10.19, 10.69, 11.2, 11.78, 12.29, 12.79, 13.38, 13.97, 14.55,
15.14, 15.73, 16.4, 16.99, 17.66, 18.33, 19, 19.67, 20.43, 21.1, 21.86, 22.61, 23.37, 24.21, 24.96,
25.8, 26.64, 27.56, 28.4, 29.33, 30.25, 31.17, 32.1, 33.1, 34.11, 35.12, 36.21, 37.3, 38.39, 39.48,
40.66, 41.83, 43.01, 44.18, 45.44, 46.78, 48.04, 49.39, 50.73, 52.15, 53.58, 55.01, 56.52, 58.03,
59.54, 61.14, 62.81, 64.41, 66.09, 67.85, 69.61, 71.46, 73.31, 75.15, 77.08, 79.1, 81.11, 83.13,
85.22, 87.41, 89.59, 91.85, 94.12, 96.47, 98.9, 101.34, 103.86, 106.37, 109.06, 111.66, 114.43,
117.2, 120.05, 122.99, 126.01, 129.03, 132.14, 135.33, 138.6, 141.88, 145.32, 148.76, 152.28,
155.98, 159.67, 163.45, 167.31, 171.25, 175.28, 179.48, 183.67, 187.95, 192.4, 191.31, 188.54,
186.02, 183.67, 181.74, 179.31, 177.46, 175.53, 174.02, 172.68, 170.58, 168.73, 166.8, 164.87,
163.28, 161.52, 159.84, 158.24, 156.48, 154.89, 153.12, 151.61, 151.11, 150.44, 148.84, 146.74,
144.81, 143.3, 142.13, 140.53, 139.02, 137.85, 136.17, 134.74, 133.65, 132.31, 132.14, 132.73,
129.45, 127.19, 125.51, 124.59, 123.33, 122.07, 120.73, 119.63, 118.38, 117.12, 116.19, 117.37,
117.45, 117.12, 115.77, 113.76, 112.16, 111.24, 110.07, 108.89, 107.55, 106.21, 105.37, 103.94,
103.6, 105.03, 104.61, 103.35, 102.09, 101, 99.83, 99.41, 98.06, 97.23, 96.13, 95.21, 94.2, 93.36,
92.86, 92.78, 92.69, 91.6, 90.68, 89.76, 88.66, 88.24, 87.32, 86.4, 85.73, 84.8, 84.13, 83.21,
82.87, 83.46, 82.45, 81.61, 80.86, 80.19, 79.6, 78.17, 77.42, 78.01, 76.16, 75.24, 74.73, 74.48,
74.73, 73.81, 73.31, 72.38, 71.79, 71.21, 70.7, 70.12, 69.53, 68.94, 68.44, 67.93, 67.43, 67.26,
67.01, 66.76, 66, 65.42, 64.83, 64.24, 63.99, 63.32, 62.9, 62.39, 61.89, 61.47, 61.05, 61.05,
60.63, 60.3, 59.88, 59.37, 58.87, 58.53, 58.03, 57.53, 57.19, 57.02, 56.27, 55.85, 55.68, 55.43,
55.26, 54.92, 54.59, 54.09, 53.83, 53.41, 53.08, 52.66, 52.24, 52.07, 51.57, 51.32, 50.98, 50.98,
50.73, 50.22, 50.22, 49.64, 49.13, 49.05, 48.63, 48.29, 48.04, 47.71, 47.54, 47.12, 46.87, 46.62,
46.7, 46.45, 46.36, 45.69, 45.44, 45.19, 44.94, 44.6, 44.27, 44.01, 43.85, 43.59, 43.34, 43.09,
42.67, 42.75, 42.67, 42.5, 42.17, 42.0, 41.66, 41.41, 41.08, 40.82, 40.74, 40.24, 39.98, 39.48,
39.4, 39.57, 39.31, 39.23, 39.06, 38.81, 38.81, 38.31, 38.14, 37.97, 37.8, 37.55, 37.3, 36.96,
36.96, 36.71, 36.63, 36.29, 27.86, 21.02, 15.41, 7.19, 0.00, 0.00, 0.00, 0.00, 0.00, 0.00, 0.00,
0.00, 0.00, 0.00, 0.00, 0.00, 0.00, 0.00, 0.00, 0.00, 0.00, 0.00, 0.00, 0.00, 0.00, 0.00, 0.00, 0.00,
0.00, 0.00, 0.00, 0.00, 0.00, 0.00, 0.00, 0.00, 0.00, 0.00, 0.00, 0.00, 0.00, 0.00, 0.00, 0.00, 0.00,
0.00, 0.00, 0.00, 0.00, 0.00, 0.00, 0.00, 0.00, 0.00, 0.00, 0.00, 0.00, 0.00, 0.00

The converted pressure values from the pressure sensor start from 0 mmHg. The air pump starts and the bladder pressure ramps up to 192.4 mmHg well above the systolic pressure of a normal person. The air pump is stopped and the pressure is in the bladder drops as the air flows out of the orifice. The underlined pressures indicate a possible pressure increase. Once the bladder pressure drops to about 37 mmHg, the system can open the pressure relief valve to vent the bladder pressure to 0.00 mmHg. This data can be used to obtain constants used to calculate the systolic and diastolic pressures.

Each constant used in the calculation itself (systolic %, diastolic %, systolic fixed threshold, and diastolic fixed threshold, bad peak count) were determined via internal trials at a local clinic. As the quantity of samples increases, these can be adjusted to allow the device even better accuracy. The ARRAY_SIZE is the number of elements that make up the graph. A pressure reading is added every 50 ms. so the current value of 1,250 creates a graph containing pressure readings over 62.5 seconds.

The measurements from the pressure sensor can be raw pressure sensor values that correspond to pressure in units of mmHg. For example, a raw pressure value from the pressure sensor of 675 for the pressure in the bladder can correspond to a true pressure of 38 mmHg when the raw pressure value is applied to the equation above. Similarly, a raw pressure value of 3,058 equals 232 mmHg and a raw pressure value of 3,296 equals 251 mmHg.

The system can use the minSystolicAmplitude and minDiastolicAmplitude values to filter the detected peak magnitudes. If a detected peak has a magnitude that is smaller than the minSystolicAmplitude or minDiastolicAmplitude, this peak is not considered a pulse during the data processing. Currently, both minSystolicAmplitude and minDiastolicAmplitude are initially set to 5.

The MAX_BAD_PEAK_COUNT is a predetermined value, which cannot be exceeded. Once there has been MAX_BAD_PEAK_COUNT pulses in a row that are not considered valid, the system can stop processing the graph. The MAX_BAD_PEAK_COUNT can be set to 6. The TIMER_PER_MINUTE Holds the number of samples taken every minute; Currently set to 60*1000/50 or 1,200. The LOG_MODULE_X/LOG_TYPE_X is used by the log and journal functions to indicate the type of data to be logged (or stored in the journal).

In an embodiment, the inventive system processor can be configured perform various functions at specific interval periods of time. For example in an embodiment, the functions can perform calculations every 256 ms. Table 3 below list a plurality of exemplary functions.

TABLE 3

| Function | Function Description |
|---|---|
| getMaxAmplitutude( ) | searches the graph for the pulse with the |
| | second largest amplitude |
| putJournal( ) | logs values for later debug/analyses |
| LOG_uint8(module, type, | Logs values in the EEPROM |
| value) | |
| ARRAY_getCount(start, | Returns the number of indexes between |
| end, size) | start and end in a circular array. |
| value2mmHg(value) | Converts the raw pressure sensor value |
| | into bladder pressure readings in units |
| | of mmHg. |

The function, geiMax Amplitutude( ) is a function that can search the graph for the pulse with the second largest amplitude. Since there should be several pulses near the largest amplitude, this function searches for the pulse with the 2nd largest amplitude just in case there was an erroneous and extremely large pulse that should not be considered. The function putJournal( ) can log values for later debug/analyses. The putJournal( ) function can normally be disabled unless the DEBUG compiler flag is activated. The function LOG_uint8(module, type, value) logs values in the EEPROM. In this implementation (uint8), 3 bytes are stored in the log. When reading the log, the module and type bytes decide how long and the format of value. The function ARRAY_getCount(start, end, size) returns the number of indexes between start and end in a circular array. The function value2 mmHg(value) converts the raw pressure sensor value into pressure values in mmHg units.

As discussed, the system can perform processing to remove or filter erroneous pulses. In an embodiment, a fixed threshold is utilized to prevent the algorithm from passing tiny detected pulses that are below the threshold value during the entire blood pressure reading process. If the calculated logarithmic threshold is less than a predetermined value, the predetermined value will be used instead effectively dropping back to a linear scale. The code allows for a different threshold for the systolic and the diastolic pulses.

Once the threshold has been determined for a given pressure graph, each peak is evaluated to decide if the detected pulse is a valid pulse. For accurate blood pressure readings, the blood pressure monitor must choose which pulses are valid. As the bladder pressure decreases over time, a pulse is detected by a slight and temporary increase in pressure. However, there are many other actions that can cause this too. For example, if the patient moves their limb, the muscle flexes and presses against the bladder. This type of an invalid pulse can be detected by the pressure sensor but may be removed from the data used to calculate the systolic and diastolic pressures.

The first valid pulse in the graph is the systolic blood pressure reading. The system can also be used to determine if a pulse is valid or not while searching for the diastolic reading. Each subsequent valid pulse might be the diastolic reading so the location of the most recent peak must be maintained.

Because our pressure sensor can detect the smallest increase, we can detect a pulse well below 40 mmHg. So, at some point, we need to determine if the pulse is too small that a human would not have heard it. Once there have been enough bad peaks in a row, the system can identify the last valid pulse as the diastolic blood pressure. After this determination, the graph is no longer evaluated and the bladder pressure relief valve is opened and the bladder pressure is vented to ambient pressure and the bladder can be removed from the user.

The inventive system can calculate the patient's blood pressure and determine if the pulse, systolic and diastolic values are within the expected range of potential values, which can be predetermined values. For example the predetermined value range can be between 70 and 240 for the systolic and between 40 and 120 for the diastolic. This can be a final check performed before the pulse, systolic and diastolic values are transmitted to a visual display module. If the systolic or diastolic values are outside the predetermined ranges, the system can prevent these values from being displayed and instruct the user to retry the blood pressure testing.

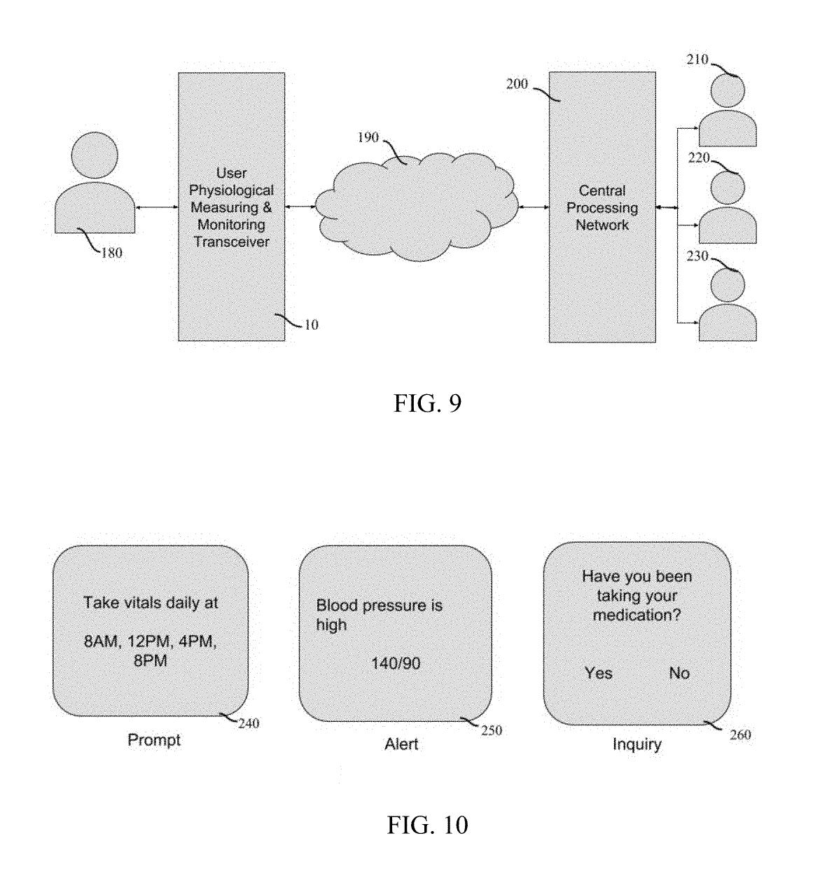

The user data obtained by the inventive system can be used and distributed as necessary. FIG. 9 provides a block diagram for one embodiment of the present invention where the transceiver device 10 has connectivity for data transmission and bi-directional communication with a central processing network 200, the central processing network technical support staff 210, the user's physician 220 and emergency services provider 230. The user 180 takes physiological measurements, which are stored in the transceiver device 10. The transceiver device 10 securely uploads the data to a cloud 190. The data is securely downloaded from the cloud 190 to a central processing network 200. Measurement data may be transmitted from the transceiver device 10 by one or more wireless and wired means, including but not limited to, a cloud, WiFi, Bluetooth, ZigBee, 2G, 3G, 4G, LTE, NFC, Sigfox, LoRaWan and telephone networks, fiber-optics and cable. The data is analyzed at the central processing network 200 by one or more means, including but not limited to, software algorithms and a qualified technical support staff 210. The data can also be transmitted to a user's physician 220 and emergency medical service provider 230. The user data of physiological conditions may be compared to thresholds established by the user's physician, the technical support staff and accepted medical standards. The central processing network 200 algorithms, technical support staff 210, the user's physician 220 and emergency services providers 230 can send prompts, alerts and inquires to the user 180 through the central processing network 200, the cloud 190 and the transceiver device 10.

FIG. 10 depicts examples of a prompt 240, alert and warning 250 and inquiry 260 in one embodiment of the present invention. Prompts, alerts and inquiries may be generated by one or more means, but not limited to; automated messaging created by algorithms contained in the transceiver device 10 and the central processing network 200 and/or messaging manually created by the central processing technical support staff 210, the user's physician 220 and emergency service providers 230. Prompts, alerts and warnings and inquiries may be comprised of one or more formats including images, text, video or audio. Prompts are generally instructions to the user 180. Alerts are generally warnings about the user's physiological measurement data delivered to the user 180, the central processing network 200, the central processing network technical support staff 210, the user's physician 220 and emergency service providers 230. Inquiries are generally questions to the user 180 resulting from the user's physiological measurement data.

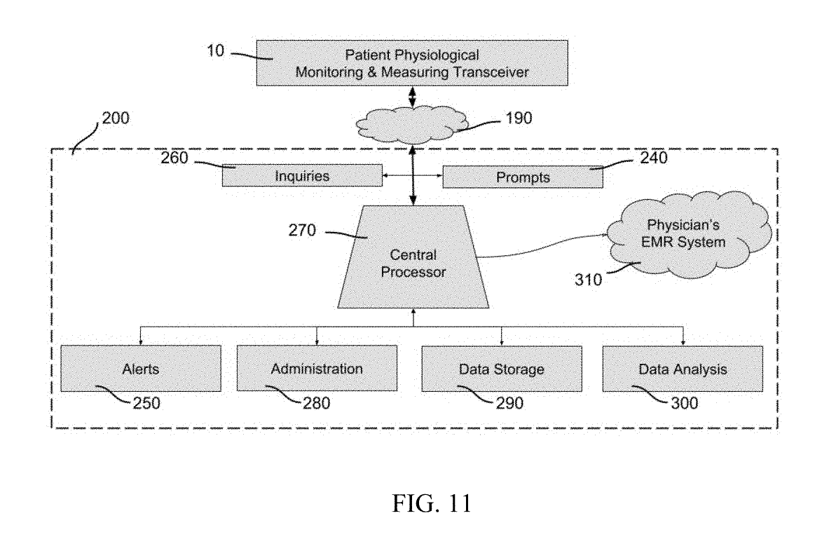

FIG. 11 provides a block diagram of one embodiment of the present invention where the transceiver device 10 has connectivity through a cloud 190 to the central processing network 200 that includes a central processor 270. The central processor 270 containing software for the execution of user data analysis 300, user data storage 290, user data administration 280, data transmission to the user physician's EMR system 310, alerts 250, prompts 240 and inquiries 260. The central processor 270 in combination with application software for data storage 290, data analysis 300 and administration 280 is also used for one or more business processes, but not limited to, generation of metrics regarding the central processing network 200 user population, user satisfaction, trends, accumulation of machine and human hours and cost analysis.

All references cited herein are intended to be incorporated by reference. Although the present invention has been described above in terms of specific embodiments, it is anticipated that alterations and modifications to this invention will no doubt become apparent to those skilled in the art and may be practiced within the scope and equivalents of the appended claims. More than one computer may be used, such as by using multiple computers in a parallel or load-sharing arrangement or distributing tasks across multiple computers such that, as a whole, they perform the functions of the components identified herein; i.e. they take the place of a single computer. Various functions described above may be performed by a single process or groups of processes, on a single computer or distributed over several computers. Processes may invoke other processes to handle certain tasks. A single storage device may be used, or several may be used to take the place of a single storage device. The present embodiments are to be considered as illustrative and not restrictive, and the invention is not to be limited to the details given herein. It is therefore intended that the disclosure and following claims be interpreted as covering all such alterations and modifications as fall within the true spirit and scope of the invention.

What is claimed is:

1. A method for measuring blood pressure comprising:
providing an air pump, a bladder, a pressure sensor, a transmitter, and an orifice;
placing a bladder around a limb of a user;
turning the air pump on to inflate the bladder and increase the pressure within the bladder with air while the air flows out of the bladder through the orifice;
increasing the pressure in the bladder until a high predetermined pressure is reached and blood flow through an artery in the limb stops while air flows out of the bladder through the orifice;
stopping the air pump and decreasing the pressure in the bladder as the air flows out of the bladder through the orifice while continuously detecting the pressures in the bladder with the pressure sensor;
transmitting the pressures in the bladder as the air flows out of the bladder detected by the pressure sensor through the transmitter to a mobile computing device;
storing in a memory of the mobile computing device, the pressures during the decreasing of the pressure in the bladder;
analyzing the pressures by the mobile computing device during the decreasing of the pressure in the bladder stored in a memory of the mobile computing device to identify a systolic pressure when a temporary pressure increase is first detected by the pressure sensor and identify a diastolic pressure when the temporary pressure increase is no longer determined to be valid by the mobile computing device; and
outputting the systolic pressure and the diastolic pressure by the mobile computing device.

2. The method for measuring blood pressure of claim 1 wherein the high predetermined pressure is between 180 mmHg and 240 mmHg.

3. The method for measuring blood pressure of claim 1 wherein rate of air flow through the orifice is greater than Mach 0.5 when the air pump is on.

4. The method for measuring blood pressure of claim 1 wherein rate of air flow through the orifice is less than Mach 0.7 when the air pump is on.

5. The method for measuring blood pressure of claim 1 further comprising:
displaying the diastolic pressure and the systolic pressure on a visual display.

6. The method for measuring blood pressure of claim 1 further comprising:
detecting a time period between the pressure increases detected by the pressure sensor while the air pump is stopped and determining a heart rate based upon the time period between the pressure increases; and
outputting the heart rate by the mobile computing device.

7. The method for measuring blood pressure of claim 1 wherein a time period between the pump stopping and detecting the systolic pressure is less than 4 seconds.

8. The method for measuring blood pressure of claim 1 further comprising:
providing a pressure release valve coupled to the bladder;
closing the pressure release valve while the air pump is on, the systolic pressure is detected, and the diastolic pressure is detected; and
opening the pressure release valve after the temporary pressure increase is no longer detected to be valid by the mobile computing device.

9. The method for measuring blood pressure of claim 1 wherein the systolic pressure and the diastolic pressure are displayed on a visual display of the mobile computing device.

10. The method for measuring blood pressure of claim 1 further comprising:
analyzing by the mobile computing device, the pressure in the bladder during a predetermined time period from when the high predetermined pressure is reached for criteria representing the systolic pressure.

11. A method for measuring blood pressure comprising:
providing an air pump, a bladder, a pressure sensor, a processor, a memory, and an orifice, wherein the air pump, pressure release valve and orifice are coupled to the pressure sensor and the pressure sensor and memory are in communication with the processor;
placing a bladder around a limb of a user;
turning the air pump on to inflate the bladder and increase the pressure within the bladder with air while air flows out of the bladder through the orifice;
increasing the pressure in the bladder until a high predetermined pressure is reached and blood flow through an artery in the limb stops;
stopping the air pump when the predetermined pressure is reached;
decreasing the pressure in the bladder as the air flows out of the bladder through the orifice while measuring the pressure with the pressure sensor;
recording by the processor and the memory the pressures in the bladder during a predetermined time period from when the predetermined pressure is reached;
analyzing by the processor, the pressure in the bladder during a predetermined time period from when the high predetermined pressure is reached for criteria representing the systolic pressure;
detecting a systolic pressure when a temporary pressure increase is first detected by the pressure sensor while the air pump is stopped; and
detecting a diastolic pressure when the temporary pressure increase is no longer determined to be valid by the processor.

12. The method for measuring blood pressure of claim 11 wherein the high predetermined pressure is between 180 mmHg and 240 mmHg.

13. The method for measuring blood pressure of claim 11 wherein rate of air flow through the orifice is greater than Mach 0.5 when the air pump is on.

14. The method for measuring blood pressure of claim 11 wherein rate of air flow through the orifice is less than Mach 0.7 when the air pump is on.

15. The method for measuring blood pressure of claim 11 further comprising:
displaying the diastolic pressure and the systolic pressure on a visual display coupled to the processor.

16. The method for measuring blood pressure of claim 11 further comprising:
detecting a time period between the pressure increases detected by the pressure sensor while the air pump is stopped and determining a heart rate based upon the time period between each of the temporary pressure increases.

17. The method for measuring blood pressure of claim 11 wherein a time period between the pump stopping and detecting the systolic pressure is less than 4 seconds.

18. The method for measuring blood pressure of claim 11 further comprising:
providing a pressure release valve coupled to the bladder;
closing the pressure release valve while the air pump is on, the systolic pressure is detected, and the diastolic pressure is detected; and
opening the pressure release valve after the diastolic pressure is detected.

19. The method for measuring blood pressure of claim 11 further comprising:
providing a blood oximeter coupled to the processor;
placing the blood oximeter on a portion of the user;
detecting by the blood oximeter, a blood oxygen level of the user; and
displaying the blood oxygen level on a visual display coupled to the processor.
